(19) United States
(12) Patent Application Publication
(10) Pub. No.: US 2026/0248543 A1
(43) Pub. Date: Aug. 27, 2026

(54) RIB FIXATION SYSTEMS, METHODS, AND DEVICES

(71) Applicant: ThoraGenix Innovations, Inc., Tampa, FL (US)

(72) Inventors: Frederick J. Thabet, Boca Raton, FL (US); Ravi Kanagala, Boca Raton, FL (US); Keri George, Calgary (CA)

(73) Assignee: ThoraGenix Innovations, Inc., Tampa, FL (US)

(21) Appl. No.: 19/345,295

(22) Filed: Sep. 30, 2025

Related U.S. Application Data
(60) Provisional application No. 63/701,218, filed on Sep. 30, 2024.

(51) Int. Cl. A61B 17/82; A61B 17/56; A61B 17/86; A61L 27/24; A61L 27/54

(52) U.S. Cl. CPC A61B 17/823; A61B 17/86; A61L 27/24; A61L 27/54; A61B 2017/564; A61L 2430/02

(57) ABSTRACT

Systems, methods, and devices disclosed herein include a rib fixation device with an elongated body having a first sidewall and a second sidewall. A hinge is formed into a top portion of the elongated body such that the first sidewall is separable from the second sidewall to form a gap at a bottom portion of the elongated body. Additionally, the rib fixation device includes one or more dimension(s), such as a width dimension of the elongated body, which corresponds to one or more dimension(s) of a target rib, such that the elongated body is operable for placement around a damaged portion of the target rib. The device also includes a tightening mechanism (e.g., a threaded screw) for bringing the first sidewall and the second sidewall together during a placement procedure. Furthermore, the elongated body includes one or more portal(s) to receive a bone growth regenerative putty and/or bicortical screws.

### CROSS REFERENCE TO RELATED APPLICATION

This application claims priority to U.S. Provisional Patent Application Ser. No. 63/701,218, filed Sep. 30, 2024, and titled “RIB FIXATION SYSTEMS, METHODS, AND DEVICES,” which is incorporated by reference herein in its entirety.

### BACKGROUND

Rib fractures occur for a multiple of reasons including car accidents, sports injuries, and falls. Lots of different break patterns can occur, such as a stress fracture, an avulsion fracture, a comminuted fracture, and a floating fracture. People with osteoporosis are even more likely to experience rib fractures. The most serious rib fracture can cause damage to internal organs including the lungs, liver, kidneys, heart, and spleen. These injuries typically require surgery to treat the rib fracture, which involves implanting plates, screws, pins, and/or wires into the patient to hold the pieces of bone together. The unique shape of the ribs makes this process difficult and can cause multiple complications, such as malunion or nonunion of the bones and/or bone infections.

It is with these observations in mind, among others, that various aspects of the present disclosure were conceived and developed.

### SUMMARY

Systems, methods, and devices disclosed herein can address the aforementioned problems. For instance, a rib fixation system can include an elongated body having a first sidewall and a second sidewall. A hinge can be formed into a top portion of the elongated body such that the first sidewall is separable from the second sidewall to form a gap at a bottom portion of the elongated body. Moreover, the system can include a width dimension of the elongated body corresponding to a dimension of a target rib, such that the elongated body is operable for placement around a damaged portion of the target rib.

In some examples, the elongated body can be made of a material with a thickness value at the top portion such that the hinge includes a living hinge formed from the material. The system can also include a screw plate having one or more screw openings formed adjacent to a bottom edge of the first sidewall. Additionally, the system can have a base plate disposed at a bottom edge of the second sidewall, opposite the screw plate, and operable to receive an end of a screw passing through the screw plate. Furthermore, the system can include a regenerative bone growth putty operable for positioning at the damaged portion of the target rib. The regenerative bone growth putty can include at least one of a bovine collagen matrix, a bioglass, or a bone growth compound. The system can also include one or more portals, formed into the elongated body, operable to receive the regenerative bone growth putty via injection through the one or more portals. One or more bicortical screws can be operable for insertion into the one or more portals. Additionally, the system can include a tightening mechanism, disposed on the elongated body and operable to, upon being actuated, bring the first sidewall and the second sidewall together; and/or secure the first sidewall in place relative to the second sidewall.

In some instances, a rib fixation device can include an elongated body with a curved outer surface defining a first sidewall and a second sidewall. The device can also include a hinge formed into the elongated body such that the first sidewall is movable relative to the second sidewall. Furthermore, the device can have a width dimension of the elongated body corresponding to a dimension of a target rib, such that the elongated body is operable for placement around a damaged portion of the target rib.

In some scenarios, the hinge can be a living hinge running along a length dimension of the elongated body. A first edge of the first sidewall can be spaced apart from a second edge of the second sidewall forming a gap between the first edge and the second edge. Also, the elongated body can include an at least partially transparent sidewall portion. Moreover, the device can include a tightening screw disposed on the elongated body operable to, upon actuation, move the first sidewall relative to the second sidewall. Furthermore, the device can include one or more portals operable to receive an injection of a regenerative bone growth putty, and/or an insertion of a bicortical screw.

In some examples, a method of rib fixation includes placing an elongated body of a rib fixation device around a damaged portion of a target rib. The method can also include bringing a first sidewall of the elongated body closer to a second sidewall of the elongated body. Additionally, the method can include securing, using a locking mechanism formed into the elongated body, a position of the first sidewall relative to the second sidewall.

In some instances, the bringing of the first sidewall to the second sidewall can include actuating a living hinge formed along a top portion of the elongated body. The securing of the position of the first sidewall relative to the second sidewall can cause a regenerative bone growth putty to be applied to and/or secured in place at the damaged portion. For instance, a user can place the regenerative bone growth putty on an interior surface of the sidewall(s) and/or onto the damaged portion prior to securing the elongated body in place. Also, the method can include performing one or more manufacturing operations to create the elongated body which can include laser cutting a piece of titanium. Additionally or alternatively, the method can include injecting a regenerative bone growth putty into one or more portals formed into the elongated body. Moreover, the method can include inserting one or more bicortical screws into the one or more portals to secure the rib fixation device around the damaged portion of the target rib.

### BRIEF DESCRIPTION OF THE DRAWINGS

FIG. 1 depicts an example system including a rib fixation device positioned over a damaged portion of a target rib.

FIG. 2 depicts an example system including a rib fixation device including a screw plate and a base plate.

FIG. 3A depicts a top view of an example system including a rib fixation device with a tightening mechanism and one or more portals.

FIG. 3B depicts a bottom view of an example system including a rib fixation device with a first sidewall and a second sidewall in a closed configuration.

FIG. 3C depicts a side view of an example system including a rib fixation device having a thickness which defines a living hinge.

FIG. 4 depicts an example method to heal a rib using a rib fixation device, which can be performed by any of the system(s) depicted in FIGS. 1-3C.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The systems, methods, and devices disclosed herein include a rib fixation device with specialized components to treat a rib fracture easily and effectively resulting in improved patient outcomes. Additionally or alternatively to treating a rib fracture, the technology disclosed herein can be used to treat damaged bone, degenerated bone, demineralized bone, genetically deformed bone, idiopathically deformed bone, infected bone, iatrogenically destabilized bone, traumatized bone, and/or combinations thereof. Furthermore, although rib fractures are primarily discussed herein, other types of bone can be treated, such as other portions of the thoracic skeletal system and/or other skeletal system(s).

In some examples, the rib fixation device can include an elongated body having a first sidewall and a second sidewall. These sidewalls can be separable during a placement procedure during which the rib fixation device is placed over the damaged portion of the rib. For instance, a hinge can be formed into a top portion of the elongated body which is used to open and/or close a gap at a bottom portion of the elongated body. A threaded screw-type tightening mechanism can also be used to bring the sidewalls together and secure them in place once the rib fixation device has been oriented by the surgeon. Furthermore, the elongated body can include one or more portal(s) to receive a bone growth regenerative putty and/or bicortical screws. With multiple dimensions corresponding to the dimensions of a target rib, such as a width dimension of the elongated body, the rib fixation device can provide improved structural support for healing the rib while holding the bone growth regenerative putty in place. As such, the systems disclosed herein can decrease the amount of time to perform the surgical procedure, and decrease the healing time post-operation.

Additional benefits and advantages of the disclosed technology will become apparent from the detailed description below.

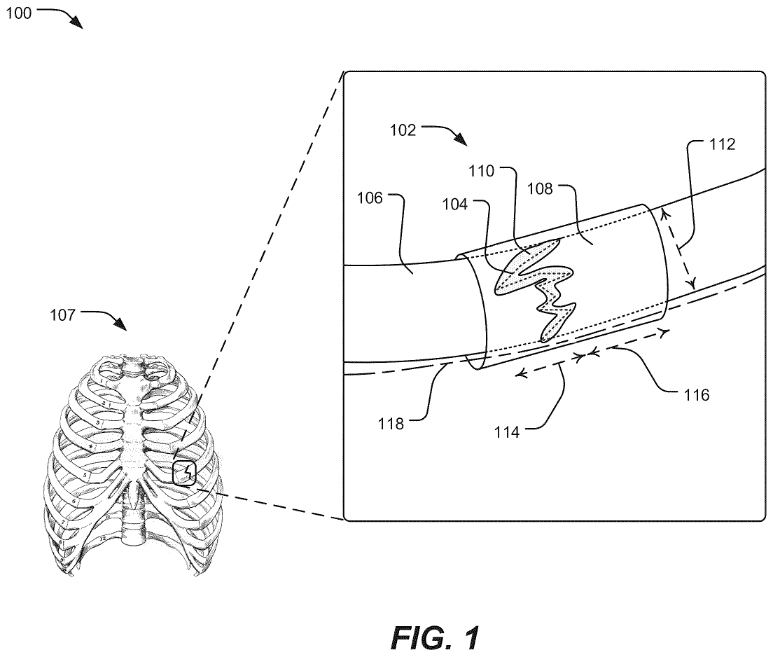

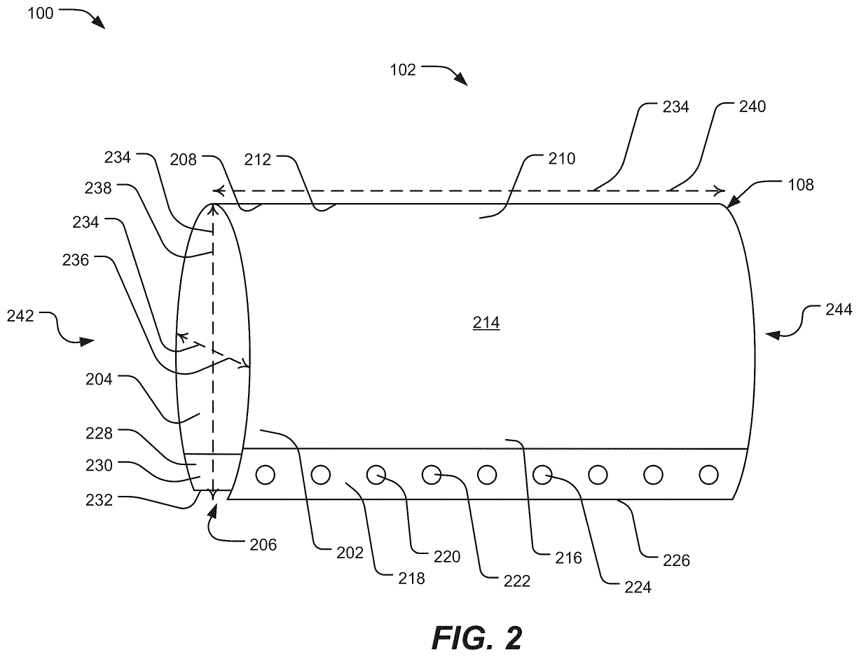

FIGS. 1 and 2 illustrate an example system 100 including a rib fixation device 102 for placement around a damaged portion 104 of a target rib 106 and/or other portion(s) of the thoracic skeletal system 107. The rib fixation device 102 can include an elongated body 108 having a first sidewall 202 separable from a second sidewall 204 to form a gap 206. During a placement procedure, the target rib 106 can be slid through the gap 206 such that the rib fixation devices is positioned 102 at a location corresponding to the damaged portion 104.

Additionally, in some instances, the system 100 can include a regenerative bone growth putty 110, which can be placed into and/or around the damaged portion 104 of the target rib 106. The regenerative bone growth putty 100 can be formed of a matrix material, such as demineralized bone matrix (DBM) and/or a collagen matrix (e.g., a bovine collagen matrix), and/or a bioglass. These components can make the regenerative bone growth putty 110 conductive and/or inductive such that cells (e.g., blood cells) are drawn into the regenerative bone growth putty 110. Moreover, a composition of the regenerative putty 110 may be irrigation-resistant with respect to blood. The regenerative bone growth putty 110 can include various bone growth compounds (e.g., osteoblasts, stem cells, exosomes, and so forth) as well as an adhesive filler (e.g., mollusk-based glue). Additionally or alternatively, the components of the regenerative bone growth putty 110 can be included in a regenerative bone growth liquid, a regenerative bone growth gel, a regenerative bone growth powder, regenerative bone growth granules, or so forth.

Turning to FIG. 2, the rib fixation device 102 can include a hinge 208 formed along a top portion 210 of the elongated body 108. The hinge 208 can be a living hinge 212 formed by the material 214 which comprises the elongated body 108. For instance, the elongated body 108 and/or at least the top portion 210 of the elongated body 108 can have a thickness value 302 such that the top portion 210 is flexible and/or bendable. By flexing/bending at the top portion 210, the first sidewall 202 can be separable from the second sidewall 204 to form and/or increase a size of the gap 206 at a bottom portion 216 of the elongated body 108, so that the elongated body 108 can be slid over the target rib 106. Additionally, once the rib fixation device 102 is in place over the target rib 106, the living hinge 212 can be used to bring the first sidewall 202 and the second sidewall 204 back together, closing and/or reducing the gap 206.

In some examples, the rib fixation device 102 can include a screw plate 218 formed into the elongated body 108, for instance, at the bottom portion 216. The screw plate 218 can include one or more screw holes 220, such as a plurality of screw holes 222 arranged in a row 224. The plurality of screw holes 222 can be disposed adjacent to a first bottom edge 226 of the first sidewall 202 and/or the second sidewall 204. The screw plate 218 can include an additional layer of material formed into and/or layered onto the material 214 which forms the elongated body 108. Additionally or alternatively, the screw plate 218 can be at the bottom portion of the first sidewall 202 and/or the second sidewall 204 without any additional material. In other words, the plurality of screw holes 222 can be formed directly into the first sidewall 202 and/or the second sidewall 204. Moreover, the one or more screw holes 220 can be formed at other locations on the elongated body 108, such as anywhere along the first sidewall 202 and/or the second sidewall 204, at the top portion 210, at combinations thereof, and so forth.

In some scenarios, the rib fixation device 102 can include a base plate 228, which can be formed into the first sidewall 202 and/or the second sidewall 204. The base plate 228 can include an additional layer 230 of material designed to receive an end of one or more screws passing through the one or more screw hole(s) 220. For instance, the base place 228 can be disposed adjacent to a second bottom edge 232 of the first sidewall 202 and/or the second sidewall 204 opposite from the screw plate 218, that is, across the gap 206 from the screw plate 218. During a securement procedure, the one or more screw holes(s) 220 can be aligned with the base plate 228 (e.g., and/or can overlap with the base plate 228), and one or more screws, such as a plurality of screws, can be threaded through the screw hole(s) 220. The base plate 228 can operate as a stop or obstruction for the ends of the screws to prevent the screws from passing through the second sidewall 204. The base plate 228 can define a termination point and/or maximum depth of the screws by receiving and stopping the ends of the screws. In this way, the base plate 228 can operate to prevent unintentional puncturing of nearby tissue with the screws.

In some examples, the elongated body 108 can have one or more dimensions 234 which correspond to one or more anatomical dimension(s) 112 of the target rib 106. For instance, the elongated body 108 can have a width dimension value 236, in a closed and/or implanted configuration, which corresponds to a width dimension value (e.g., a thickness) of the target rib 106. This correspondence can include the elongated body's width dimension value 236 being a same value as the width dimension value of the target rib 106, or being slightly larger (e.g., 0.1-5 mm larger and/or 0.1%-10% larger). Additionally, the elongated body 108 can have a height dimension value 238 which can correspond to a height dimension value of the target rib 106 (e.g., by being 0.1-5 mm larger and/or 0.1%-10% larger). Moreover, the elongated body 108 can have a length dimension value 240 which can correspond to a first length dimension 114 of the damaged portion 104 of the target rib 108 (e.g., 0.1-5 mm larger and/or 0.1%-10% larger).

In some instances, the length dimension value 240 of the elongated body 108 can be provided such that the elongated body 108 covers both the first length dimension 114 of the damaged portion 104 (e.g., a fractured portion) of the target rib 106 and a second length dimension 116 of an undamaged portion (e.g., an unfractured portion) of the target rib 106. In some scenarios, a ratio of the second length dimension 116 covered by the elongated body 108 to the first length dimension 114 can vary based on a type and/or severity of the damaged portion 104, and a surgeon's discretion in how much structural support is needed by extending the elongated body 108 beyond the first length dimension 114 of the damaged portion 104.

Moreover, one or more of the dimension(s) 234 can vary from one end of the elongated body 108 to another end. This can include a varying width dimension value 236 and/or a varying height dimension value 238, such that the elongated body 108 at least partly tapers from one end to another. Additionally or alternatively, the surgeon can strategically place the rib fixation device 102 on the target rib 106 at a thicker portion and/or a stronger portion of the target rib 106 to increase the stability and/or structural support provided by the rib fixation device 102. Furthermore, one or more of the dimension(s) 234 can be adjustable prior to performing the placement procedure. For example, the elongated body 108 can have an adjustable length dimension value 240 via one or more segmentation and/or divider lines formed into the elongated body 108. The segmentation and/or divider line(s) can extend vertically along the first sidewall 202 and/or the second sidewall 204 and can include markings, perforations, and/or less/weakened material such that the elongated body 108 can be broken apart at the segmentation and/or divider line(s) to reduce the length dimension value 240. Additionally or alternatively, the elongated body 108 can have telescoping segments (e.g., with different width dimension value(s) 236 and/or height dimension value(s) 238), such that the length dimension value 240 can be increased and/or decreased by a surgeon to match a particular dimension of the target rib 106 and/or the damaged portion 104 of the target rib 106. Also, the first sidewall 202 and/or the second sidewall 204 can include curves and/or contours that match the particular shape(s) and/or contour(s) of different portions of the target rib 106 receiving the rib fixation device 102.

Additionally, in some scenarios, the rib fixation device 102 can undergo one or more reorientation operations subsequent to the placement procedure onto the target rib 106. The rib fixation device 102 can have multiple adjustment degrees of freedom on the target rib 106. For instance, the elongated body 108 can be rotatably adjustable by rotating the elongated body 108 about a center axis which runs along the length dimension value 240. Moreover, the elongated body 108 can be transversely adjustable by sliding in a direction corresponding to the length dimension value 240. Additionally, the elongated body 108 can be tiltably adjustable in multiple dimensions by tilting one end 242 of the elongated body 108 higher or lower (e.g., in the direction of the height dimension value 238) than another end 244 of the elongated body 108, and/or by titling the one end 242 closer or further from a posterior of the patient (e.g., in the direction of the width dimension value 236) than the other end 244.

Additionally, or alternatively, in some examples, the rib fixation device 102 can include “sling” or :pocket” made from mesh or another material. This type of sling can be used to capture bone grafting matrix, such as the regenerative bone growth putty 110, and can augment the other hardware (e.g., the elongated body 108) used to fixate the fractured rib to support healing. In some cases, the sling can act as a “wrap” or “sock” to circumnavigate the fractured rib, which can help with alignment of the rib. This use of the sling can also provide a means by which to hold the fracture and or pieces of the fractured bone (e.g., the target rib 106) in place and/or to help immobilize the fractured rib. Moreover, the sock or sling can be used to help establish compression on the fractured bone (e.g., the target rib 106). The materials for the sock or sling can be made from one or more of titanium, titanium mesh, prolean, surgical plastics, nylon, collagen, collagen impregnated with bone grafting minerals and growth factors, any combination thereof, and/or the same or different material as the elongated body 108 of the rib fixation device 102. Furthermore, any surgical elastic material can be used to help stabilize, compress, fixate and bind the fractured ribs. Also, the sling or sock can be used with the grafting matrix (e.g., the regenerative bone growth putty 110), it can be made from the same material as the grafting matrix, a as a component of the material makeup of the sling or sock.

Moreover, the rib fixation device 102 can include a claw-like structure (e.g., clamp) which can at least partially wrap around the fractured rib. This can include a screw set (e.g., at the top of the rib fixation device 102), which a surgeon can screw to close the clamp and/or seal the rib fixation device 102. Additionally, the rib fixation device 102 can include a bone stimulator, such as one or more electrical wires which can deliver an electrical charge at the fracture sight to stimulate bone growth. In other words, the rib fixation device 102 can include wires and/or circuitry to add electrical impulse bone stimulation to help accelerate bone growth. This bone stimulator component can recharge from outside the chest cavity, and/or remotely, for instance, once per day. Also, the bone stimulator component can include a magnet-based bone growth stimulator, and/or a vibration-based bone growth stimulator. Furthermore, the rib fixation device 102 can include various interior features, such as a claw-like structure, or other structures inside the elongated body 108 (e.g., along an interior surface of the elongated body 108) which can cause healing for the fractured rib. For example, a thin film of material (e.g., bone glass, antibiotics, growth factors) can be disposed inside the rib fixation device 102 and can be held in place against the fractured rib when the rib fixation device is clamped shut.

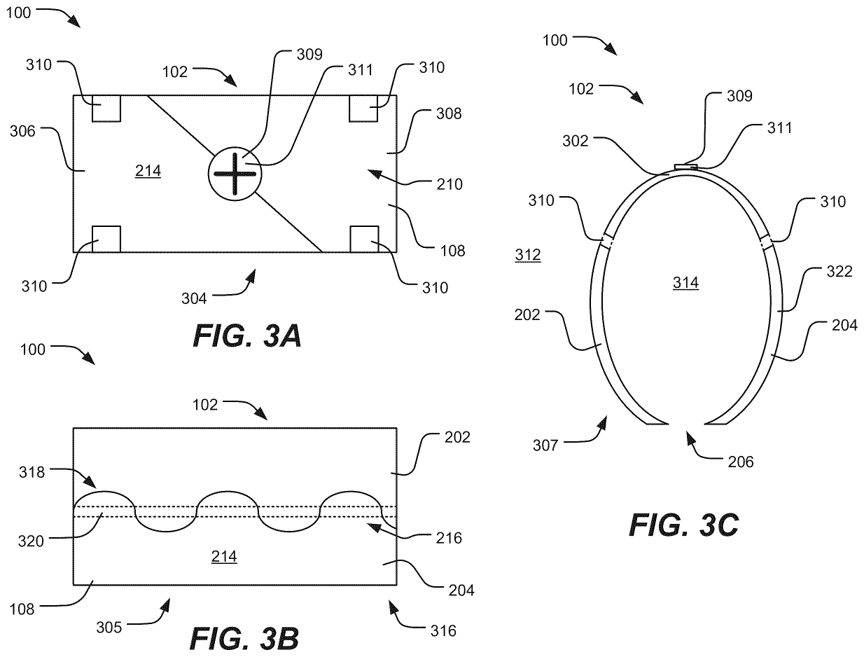

FIGS. 3A-3C depict example system(s) 100 including the rib fixation device 102, with FIG. 3A illustrating a top view 304, FIG. 3B illustrating a bottom view 305, and FIG. 3C illustrating a side view 307. The rib fixation device 102 depicted in FIGS. 3A-3C can be similar to, identical to and/or can form at least a portion of the system(s) 100 depicted in FIGS. 1 and 2.

In some examples, the rib fixation device 102 can include a tightening mechanism 309 which can be used to bring a first portion 306 of the elongated body 108 (e.g., including the first sidewall 202) together with a second portion 308 (e.g., including the second sidewall 204). For instance, the tightening mechanism 309 can include a threaded screw 311 extending from the top portion 210 of the elongated body 108, disposed between and engaging the two portions 306 and 308. Actuating the tightening mechanism 309 can bring the two portions 306 and 308 together to close the gap 206. Additionally or alternatively, actuating the tightening mechanism 309 can be used to secure (e.g., lock) the first portion in place with respect to the second portion 308. In this way, the tightening mechanism can operate as a locking mechanism. That said, in some instances, the rib fixation device 102 can include a locking mechanism which is separate from the tightening mechanism 109, such as a tab, locking screw, or bolt. An actuation of the tightening mechanism (e.g., in another direction), can also be used to release the first portion 306 from the second portion 308 and/or increase a dimension of the gap 206.

Furthermore, the rib fixation device 102 can include one or more portals 310 formed into the first sidewall 202, second sidewall 204, and/or other portion(s) of the elongated body 108. The portal(s) 310 can include openings, apertures, luer injection ports, or so forth which, provide a pathway from an exterior 312 of the elongated body 108 to an interior 314 of the elongated body 108. In some instances, the portal(s) 310 can be multi-functional. For example, the portal(s) 310 can be operable to receive an injection of the regenerative bone growth putty 110 once the rib fixation device is positioned 102 over the damaged portion 104 of the target bone 106. The portal(s) 310 can also be operable to receive bicortical screws, which can be inserted into the portal(s) 310 to further secure the rib fixation device 102 onto the target rib 106. The portal(s) 310 can include threading for receiving the bicortical screw(s). Moreover, the portal(s) 310 can include a transparent material and/or cover to form a window which provides visual access to the interior 314 of the elongated body 108. In some instances, the rib fixation device 102 can include any number of portal(s) 310 such as one, two, three, four, five, six, seven, eight, or so forth. In some scenarios, the rib fixation device 102 includes four portals 310, with two portals 310 formed into the first sidewall 202 and two portals 310 formed into the second sidewall 204. The portal(s) 310 can be circular, oval, square, rectangular, triangular, and/or an irregular shape. Additionally, the portal(s) 310 can include removable covers, which can be placed over the portal(s) 310 before and/or after use.

In some instances, the first sidewall 202 can mate with the second sidewall 204 when the rib fixation device 102 is in a closed configuration 316. The first bottom edge 226 of the first sidewall 202 can abut and/or or overlap with the second bottom edge 232 of the second sidewall 204 in the closed configuration 316, such that a dimension of the gap 206 is reduced to zero. The first bottom edge 226 and/or the second bottom edge 232 can be linear. Alternatively, the first bottom edge 226 and/or the second bottom edge 232 can form can have a wave profile 318, a zig-zag profile, a square step profile, or so forth. Additionally, the bottom portion 216 of the elongated body 108 (e.g., and/or other portion(s) such as a side portion) can include one or more channels 320 defined into the material 214 to provide additional space for blood vessels 118 at the target rib 106. The channel(s) 320 can include an elongated indentation or additional extension of the material 214 which runs along the bottom portion 216 of the elongated body 108 in the direction of the length dimension value 240. Additionally or alternatively, the channel(s) 320 can include one or more cutouts or openings to provide space for the blood vessels 118.

In some examples, the rib fixation device 102 can have a consistent thickness value 302 throughout the elongated body 108, and/or the elongated body 108 can have a consistent type of material (e.g., stainless steel, titanium, or so forth) throughout its different portions. The living hinge 212 can be formed of a same material as the first sidewall 202 and/or the second sidewall 204. Additionally or alternatively, the elongated body 108 can have a different thickness (e.g., greater or lesser) at the top portion 210 (e.g., the thickness value 302) than a thickness 322 at other portions to create the living hinge 212. Moreover, the elongated body 108 can include different materials, such as a first material transitioning to a second, different material via a gradient, and/or different sections formed of different materials, which can also be used to form the living hinge 212. For instance, the living hinge 212 can be formed of a first material which is a different material than a second material of which the first sidewall 202 and/or the second sidewall 204 are formed. Additionally, the rib fixation device 102 can include a clamping and/or gripping portion(s) extending from the elongated body 108 and/or formed into the elongated body operable to be grabbed by one or more tools, such as forceps. This can include one or more protrusions and/or textured surfaces. With the various features disclosed herein, the rib fixation device 102 can be a multi-functional device in that it can provide structural support to the damaged portion 104 of the rib 106 and/or can provide positioning functionality to maintain the regenerative bone growth putty 110 in place at the damaged portion 104.

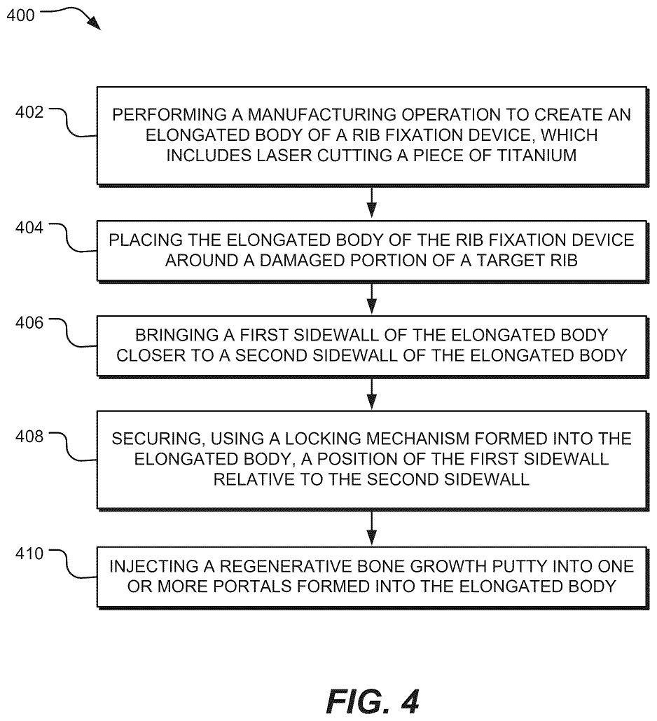

FIG. 4 depicts an example method 400 of rib fixation, which can be performed by any of the system(s) 100 disclosed herein regarding FIGS. 1-3C.

For example, at operation 402, the method 400 can perform a manufacturing operation to create an elongated body of a rib fixation device. The manufacturing operation can include laser cutting a piece of titanium. At operation 404, the method 400 can place the elongated body of the rib fixation device around a damaged portion of a target rib. At operation 406, the method 400 can bring a first sidewall of the elongated body closer to a second sidewall of the elongated body. At operation 408, the method 400 can secure, using a locking mechanism formed into the elongated body, a position of the first sidewall relative to the second sidewall. At operation 410, the method 400 can inject a regenerative bone growth putty into one or more portals formed into the elongated body.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. Any of the features or components of an implementation disclosed herein can be combined with any of the features or components of any other implementations disclosed herein. These and other variations, modifications, additions, and improvements may fall within the scope of the disclosure as defined in the claims that follow.

What is claimed is:

1. A rib fixation system comprising:
an elongated body having a first sidewall and a second sidewall;
a hinge formed into a top portion of the elongated body such that the first sidewall is separable from the second sidewall to form a gap at a bottom portion of the elongated body; and
a width dimension of the elongated body corresponding to a dimension of a target rib, such that the elongated body is operable for placement around a damaged portion of the target rib.

2. The system of claim 1,
wherein,
the elongated body is made of a material with a thickness value at the top portion such that the hinge includes a living hinge formed from the material.

3. The system of claim 1, further comprising:
a screw plate having one or more screw openings formed adjacent to a bottom edge of the first sidewall.

4. The system of claim 3, further comprising:
a base plate disposed at a bottom edge of the second sidewall, opposite the screw plate, and operable to receive an end of a screw passing through the screw plate.

5. The system of claim 1, further comprising:
a regenerative bone growth putty operable for positioning at the damaged portion of the target rib.

6. The system of claim 5,
wherein,
the regenerative bone growth putty includes at least one of a bovine collagen matrix a bioglass, or a bone growth compound.

7. The system of claim 5, further comprising
one or more portals formed into the elongated body, and operable to receive the regenerative bone growth putty via injection through the one or more portals.

8. The system of claim 7, further comprising:
one or more bicortical screws operable for insertion into the one or more portals.

9. The system of claim 8, further comprising:
a tightening mechanism disposed on the elongated body and operable to, upon being actuated:
bring the first sidewall and the second sidewall together; and
secure the first sidewall in place relative to the second sidewall.

10. A rib fixation device comprising:
an elongated body with a curved outer surface defining a first sidewall and a second sidewall,
a hinge formed into the elongated body such that the first sidewall is movable relative to the second sidewall; and
a width dimension of the elongated body corresponding to a dimension of a target rib, such that the elongated body is operable for placement around a damaged portion of the target rib.

11. The device of claim 10,
wherein
the hinge is a living hinge running along a length dimension of the elongated body.

12. The device of claim 11,
wherein,
the first sidewall includes a first edge,
the second sidewall includes a second edge,
the first edge is spaced from the second edge to form a gap between the first edge and the second edge.

13. The device of claim 12,
wherein,
the elongated body includes an at least partially transparent sidewall portion.

14. The device of claim 11, further comprising:
a tightening screw disposed on the elongated body operable to, upon actuation, move the first sidewall relative to the second sidewall.

15. The device of claim 10, further comprising:
one or more portals operable to receive:
an injection of a regenerative bone growth putty, and
an insertion of a bicortical screw.

16. A method of rib fixation, the method comprising:
placing an elongated body of a rib fixation device around a damaged portion of a target rib;
bringing a first sidewall of the elongated body closer to a second sidewall of the elongated body; and
securing, using a locking mechanism formed into the elongated body, a position of the first sidewall relative to the second sidewall.

17. The method of claim 16,
wherein,
the bringing of the first sidewall to the second sidewall includes actuating a living hinge formed along a top portion of the elongated body.

18. The method of claim 17,
wherein,
the securing of the position of the first sidewall relative to the second sidewall causes a regenerative bone growth putty to be applied to and secured in place at the damaged portion.

19. The method of claim 16, further comprising:
injecting a regenerative bone growth putty into one or more portals formed into the elongated body.

20. The method of claim 19, further comprising:
inserting one or more bicortical screws into the one or more portals to secure the rib fixation device around the damaged portion of the target rib.
